(19) United States
(12) Patent Application Publication
(10) Pub. No.: US 2014/0087330 A1
(43) Pub. Date: Mar. 27, 2014

(54) DENTAL RETRACTION CORD FACILITATING ABSORPTION AND BLOOD CLOTTING

(71) Applicants: John J. DISCKO, Trumbull, CT (US); William B. Dragan, Easton, CT (US)

(72) Inventors: John J. DISCKO, Trumbull, CT (US); William B. Dragan, Easton, CT (US)

(21) Appl. No.: 14/035,620

(22) Filed: Sep. 24, 2013

Related U.S. Application Data
(60) Provisional application No. 61/705,482, filed on Sep. 25, 2012.

(51) Int. Cl. A61C 9/00; A61K 6/00

(52) U.S. Cl. CPC A61C 9/0033; A61K 6/0088
USPC 433/136; 433/215

(57) ABSTRACT

A retraction cord for use in a dental procedure for widening the gingival sulcus containing kaolin. The kaolin acts as a hemostatic agent and an absorber or hydrophilic agent to reduce bleeding and dry the gingival sulcus. A binder may be used to aid in retention of the kaolin on the fibers of the retraction cord. A lubricant or release agent may be used preventing blood clots from sticking to the retraction cord upon removal. Kaolin is affected and has no adverse side effects.

### RELATED APPLICATION

This application claims the benefit of U.S. Provisional Application No. 61/705,482 filed Sep. 25, 2012.

### FIELD OF THE INVENTION

The present invention relates in general to dental procedures, and particularly to a retraction cord for retracting of gum tissue.

### BACKGROUND OF THE INVENTION

Retraction cord is often used to push or pack between gingival tissue and the margin of a prepared tooth prior to taking an impression of the prepared tooth in a dental procedure for making a crown or other dental device. This region is often called the sulcus. A dental instrument or tool is often used to push the retraction cord into the sulcus. Current retraction cords are often impregnated with an astringent or vasoconstriction agent, such as aluminum chloride, or epinephrine.

One such retraction cord is disclosed in U.S. Pat. No. 6,375,461 entitled “Gingival Retraction Cords Incorporating Propylhexedrine” issuing to Jensen et al on Apr. 23, 2013. Therein disclosed is a retraction cord including propylhexedrine for providing hemostasis. Another retraction cord is disclosed in United States Patent Application Publication No. 2011/0229849 entitled “Dental Retraction Device and Process of its Production” invented by Maurer et al and published Sep. 22, 2011. Therein disclosed are twisted staple fibers with an alginate containing compound.

Often the placement of retraction cord requires the widening and stretching of the sulcus due to physical force. This physical force may result in tissue damage and bleeding. This bleeding is not advantageous and may make a subsequent dental procedure of taking an impression of the tooth more difficult and unreliable. Typically an impression is best taken when the sulcus is clean, dry, and free of blood. The prior retraction cords that contain aluminum chloride, epinephrine, ferric sulfate, or similar chemicals generally taste and smell bad and may cause adverse reactions in some patients. Epinephrine may cause increased blood pressure or accelerated heart rate. Ferric sulfate may discolor tissue and turn black. Additionally, these materials generally do not have absorbing capabilities or are not hydrophilic. Therefore there is a need for an improved retraction cord that does not have the disadvantages associated with existing retraction cords.

### SUMMARY OF THE INVENTION

The present invention relates to a retraction cord that is used in a dental procedure to widen the sulcus. The retraction cord may be made of a twisted or braided fiber that is impregnated or coded with kaolin. The kaolin acts as a hemostatic agent to stop bleeding and as an absorbent or hydrophilic agent to dry the sulcus. A binder may be used to help retain or augment adhesion of the kaolin on the retraction cord.

Accordingly it is an object of the present invention to provide an improved retraction cord that is safe and effective.

It is an advantage of the present invention that it does not have the undesirable side effects such as unpleasant taste are darkening of the tissue as in prior retraction cords.

It is another advantage of the present invention that the sulcus is widened and dried.

It is a feature of the present invention that kaolin adheres to the fibers of the retraction cord.

It is another feature of the present invention that a binder may be used to assist in the adherence of the kaolin to the fiber of the retraction cord.

It is yet another feature of the present invention that a lubricant or release agent is used.

These and other objects, advantages, and features will become more readily apparent in view of the following more detailed description.

### BRIEF DESCRIPTION OF THE DRAWINGS

FIG. 1 schematically illustrates the placement and use of the present invention.

FIG. 2 schematically illustrates the retraction cord of the present invention.

FIG. 3 is a cross section taken along line 3-3 in FIG. 2.

FIG. 4 schematically illustrates another embodiment of the present invention.

### DETAILED DESCRIPTION OF THE PREFERRED EMBODIMENTS

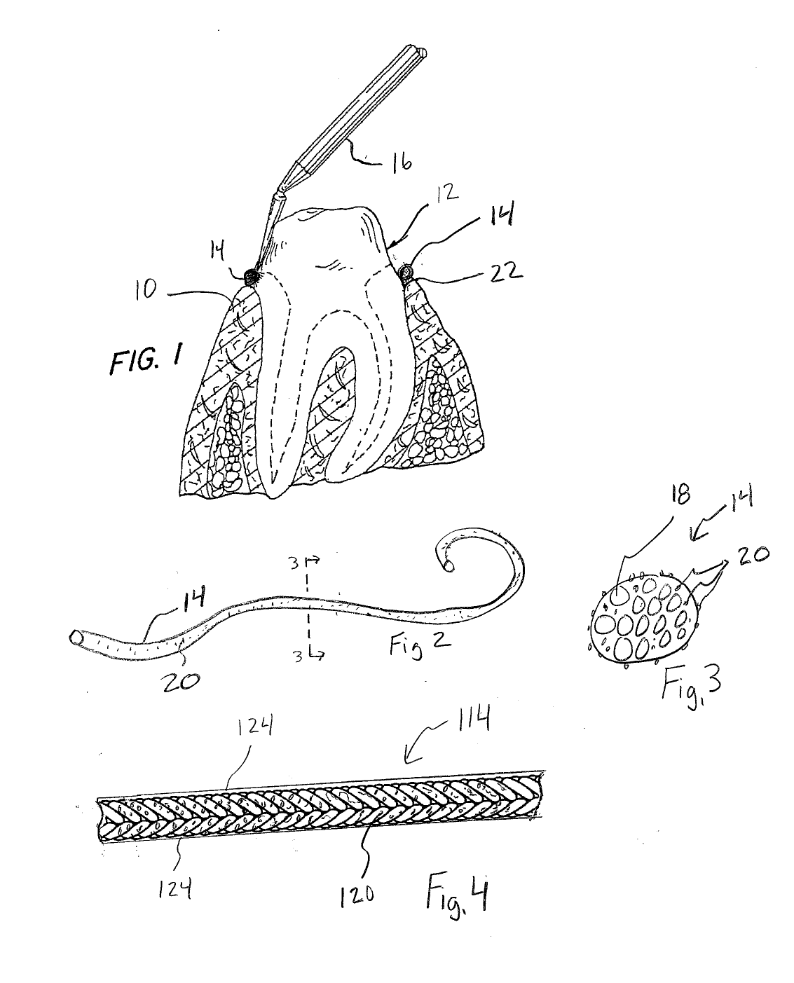

FIG. 1 schematically illustrates the application of the present invention in a dental procedure for retracting gingival or gum tissue around a prepared tooth 12. Retraction cord 14 is placed around the perimeter of a tooth 12 between the tooth surface and the gingiva 10 in a sulcus 22. The retraction cord 14 may be placed by hand and also packed with the aid of a packing tool 16. The packing tool 16 may be any blunt dental instrument that is capable of pressing on the retraction cord 14 pushing it gently down into the sulcus 22.

FIG. 2 schematically illustrates the retraction cord 14 of the present invention having a clay material, preferably kaolin, placed there on and therein. The retraction cord of the present invention may be any cord like material having fibers or strands. The fibers may be twisted or braided. Kaolin is a fine usually white clay. The kaolin may be in the form of a fine powder. Kaolin has hemostatic as well as absorbent properties.

FIG. 3 is a cross section of the retraction cord 14 illustrated in FIG. 2 taken along line 3-3 and more clearly illustrates the kaolin or clay material 20 contained on the retraction cord 14 and within the retraction cord 14 between the fibers 18. The kaolin may be placed either on the retraction cord 14 or impregnated in it, or both.

FIG. 4 illustrates another embodiment of the present invention utilizing a braided retraction cord 114. The braided retraction cord 114 has contained thereon and therein the kaolin or clay material 120. The braided retraction cord 114 may be advantageous in holding additional kaolin or clay material 120 and in exposing the kaolin or clay material 120 to the gingiva or gum surface. This embodiment also uses a lubricant or release agent coating 124 on the braided retraction cord 114. The release agent may be silicon, glycerin, or other coating material that aids in reducing friction or adherence of the braided retraction cord 114 to the gingival tissue, coagulated blood, or blood clots. This aids in placement as well as removal of the braided retraction cord 114. The lubricant or release agent 124 is particularly helpful in preventing coagulation or blood clots formed by the effects of the kaolin from sticking to the retraction cord. The kaolin clay may also be placed on the lubricant or release agent 124.

The kaolin or clay material 20 and 120 may be placed on the retraction cord 14 and 114 by any convenient method or process. For example, the kaolin or clay material may be formed on or within the retraction cord 14 and 114 by the process described in U.S. Pat. No. 7,604,819 entitled “Clay-Based Hemostatic Agents and Devices for the Delivery Thereof” issuing to Huey et al. on Oct. 20, 2009, which is herein incorporated by reference. Therein disclosed is a hemostatic device and method using a polyol and specifically glycerol as a binder to bind a clay material to a gauze substrate.

The present invention utilizing a retraction cord containing a clay material, and in particular kaolin, provides an improved retraction cord that is virtually tasteless, odorless, non-allergenic, non-staining and has no negative blood pressure or heart rate affects that are associated with other materials used in retraction cord, such as epinephrine. Additionally, the kaolin acts as a hemostatic agent and aids in blood clotting creating a clotting cascade that stops bleeding better than astringents or vasoconstrictors that simply constrict blood vessels. The kaolin does not taste terrible or bad and will not turn blood black as in some other hemostatic agents. Therefore, the present invention in utilizing retraction cord with kaolin works better than other retraction cords containing an astringent or vasoconstrictors.

While the present invention has been described with respect to several embodiments, it will be obvious that various modifications may be made without departing from the spirit and scope of this invention.

What is claimed is:

1. A retraction cord for placement in a gingival sulcus during a dental procedure comprising:
an elongated fiber material; and
kaolin placed on said elongated fiber material.

2. A retraction cord for placement in a gingival sulcus during a dental procedure as in claim 1 wherein:
said elongated fiber material is braided

3. A retraction cord for placement in a gingival sulcus during a dental procedure as in claim 1 further comprising:
a binder placed on said elongated fiber material.

4. A retraction cord for placement in a gingival sulcus during a dental procedure as in claim 3 wherein:
said binder comprises a polyol.

5. A retraction cord for placement in a gingival sulcus during a dental procedure as in claim 4 wherein:
the polyol comprises glycerol.

6. A dental retraction device for placement in a gingival sulcus during a dental procedure comprising:
a twisted fiber cord;
a binder placed on said twisted fiber cord; and
kaolin placed on said binder and said twisted fiber cord; and
a release agent placed on said twisted fiber cord,
whereby said binder facilitated said kaolin to stay in place on said twisted fiber cord and said release agent prevents blood clots from adhering to said twisted fiber cord.

7. A dental retraction device for placement in a gingival sulcus during a dental procedure as in claim 6 wherein:
said twisted fiber cord is braided.

8. A dental retraction device for placement in a gingival sulcus during a dental procedure as in claim 6 wherein:
said binder comprises a polyol.

9. A dental retraction device for placement in a gingival sulcus during a dental procedure as in claim 8 wherein:
said polyol comprises glycerol.

10. A dental retraction device for placement in a gingival sulcus during a dental procedure as in claim 6 wherein:
said release agent comprises silicon.

11. A dental retraction device for placement in a gingival sulcus during a dental procedure as in claim 6 wherein:
said release agent comprises glycerin.

12. A method of retracting gingiva forming a sulcus comprising the steps of:
impregnating a cord with kaolin clay; and
packing the cord in the sulcus in gingiva around a prepared tooth; and
removing the cord from the sulcus after the sulcus has been retracted and dried,
whereby gingiva or gum tissue is retracted and the sulcus is widened.
